(19) United States
(12) Patent Application Publication
(10) Pub. No.: US 2021/0389146 A1
(43) Pub. Date: Dec. 16, 2021

(54) INFORMATION PROCESSING APPARATUS, SUGGESTION SYSTEM, PROGRAM, AND SUGGESTION METHOD

(71) Applicant: TOYOTA JIDOSHA KABUSHIKI KAISHA, Toyota-shi (JP)

(72) Inventors: Naoki UENOYAMA, Nagoya-shi (JP); Koichi SUZUKI, Nagoya-shi (JP); Ryo KAMIMURA, Shizuoka-ken (JP); Daisuke NAKATA, Seto-shi (JP); Yuki TATSUMOTO, Nagoya-shi (JP); Yusuke FUTAKUCHI, Nisshin-shi (JP)

(73) Assignee: TOYOTA JIDOSHA KABUSHIKI KAISHA, Toyota-shi (JP)

(21) Appl. No.: 17/229,344

(22) Filed: Apr. 13, 2021

(30) Foreign Application Priority Data
Jun. 16, 2020 (JP) 2020-104112

(51) Int. Cl. G01C 21/34; G06N 5/04; G16H 20/60

(52) U.S. Cl. CPC G01C 21/3476; G16H 20/60; G06N 5/04; G01C 21/3484

(57) ABSTRACT

An information processing apparatus includes a communication unit and a control unit. The control unit is configured to determine a recipe of a dish to be suggested to a driver based on driving information on the driver of the vehicle received with the communication unit.

### INCORPORATION BY REFERENCE

The disclosure of Japanese Patent Application No. 2020-104112 filed on Jun. 16, 2020 including the specification, drawings and abstract is incorporated herein by reference in its entirety.

### BACKGROUND

### 1. Technical Field

The present disclosure relates to an information processing apparatus, a suggestion system, a program, and a suggestion method.

### 2. Description of Related Art

A system has conventionally been known which suggests a nutritionally desirable meal menu in accordance with a future action schedule of an eater (for example, Japanese Patent Application Publication No. 2016-200963).

### SUMMARY

There is room for improvement regarding suggestion of a meal menu.

An object of the present disclosure is to suggest the recipe of a dish suitable for a driver.

An information processing apparatus according to the present disclosure includes: a communication unit; and a control unit configured to determine a recipe of a dish to be suggested to a driver of a vehicle based on driving information on the driver of the vehicle received with the communication unit.

A program according to the present disclosure causes a computer to execute operation. The operation includes: receiving driving information on a driver of a vehicle; and determining a recipe of a dish to be suggested to the driver of the vehicle based on the driving information on the driver of the vehicle.

A suggestion method according to the present disclosure includes: by an information processing apparatus, receiving driving information on a driver of a vehicle; and by the information processing apparatus, determining a recipe of a dish to be suggested to the driver of the vehicle based on the driving information on the driver of the vehicle.

According to the present disclosure, it is possible to suggest the recipe of a dish suitable for a driver.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 shows configuration of a suggestion system according to an embodiment of the present disclosure;

FIG. 2 is a block diagram showing detailed configuration of the suggestion system shown in FIG. 1;

FIG. 3 shows an example of information on the recipes of dishes according to an embodiment of the present disclosure; and

FIG. 4 is a sequence diagram showing operation of the suggestion system shown in FIG. 1.

### DETAILED DESCRIPTION OF EMBODIMENTS

An embodiment of the present disclosure will be described hereinbelow with reference to the drawings. In the following component members shown in the drawings, like component members are denoted by like reference numerals.

### Configuration of Suggestion System

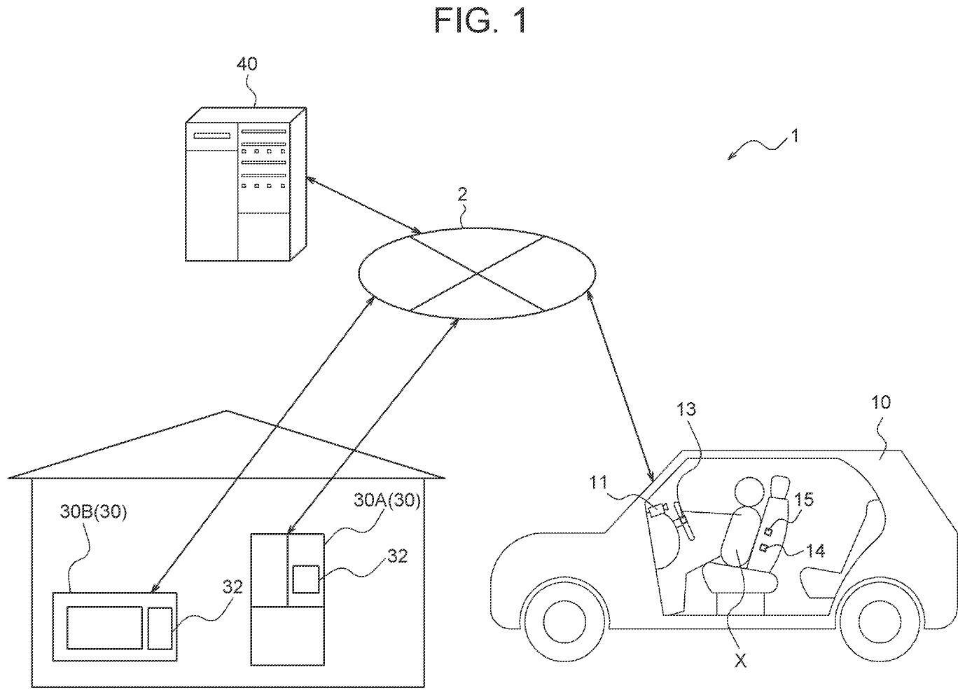

As shown in FIG. 1, a suggestion system 1 according to an embodiment of the present disclosure includes a vehicle 10, home electric appliances 30A, 30B, and an information processing apparatus 40.

Hereinafter, the home electric appliance 30A and the home electric appliance 30B are collectively stated as the “home electric appliance or appliances 30” unless otherwise being distinguished from each other. FIG. 1 shows the suggestion system 1 including two home electric appliances 30. However, the suggestion system 1 may include at least one home electric appliance 30.

The vehicle 10, the home electric appliances 30, and the information processing apparatus 40 can communicate with each other through a network 2. The network 2 may be any network including mobile communications networks, and the Internet.

The vehicle 10 can be driven by a driver X. In the present disclosure, the term “driver” refers to a person who drives the vehicle 10 on a daily basis. The vehicle 10 may be a private vehicle of the driver X.

The vehicle 10 may be any type of vehicle. Examples of the vehicle 10 include a gasoline vehicle, a diesel vehicle, a hybrid vehicle (HV), a plug-in hybrid vehicle (PHV), an electric vehicle (EV), or a fuel cell vehicle (FCV). Driving of the vehicle 10 may be automated in any level. For example, the level of automation is any one of level 1 to level 5 defined by Society of Automotive Engineers (SAE). The vehicle 10 may be a vehicle dedicated for Mobility as a Service (MaaS).

The home electric appliances 30 can be disposed in the home of the driver X. The home electric appliances 30 include output units 32. The home electric appliances 30 may be any home electric appliances as long as the home electric appliances 30 include the output units 32. The home electric appliance 30A is a refrigerator, for example. The home electric appliance 30B is a microwave oven, for example.

As will be described later, the information processing apparatus 40 determines the recipe of a dish to be suggested to the driver X. The information on the recipe of the dish determined by the information processing apparatus 40 is transmitted to the home electric appliance or appliances 30 through the network 2. The home electric appliance 30 can output the information on the recipe of the dish determined by the information processing apparatus 40 from the output unit 32. With such configuration, after going back home by driving the vehicle 10, the driver X can refer to the recipe of the dish output from the output unit 32 of the home electric appliance 30.

The information processing apparatus 40 may be a dedicated computer configured to function as a server, a general-purpose personal computer, a cloud computing system, or the like.

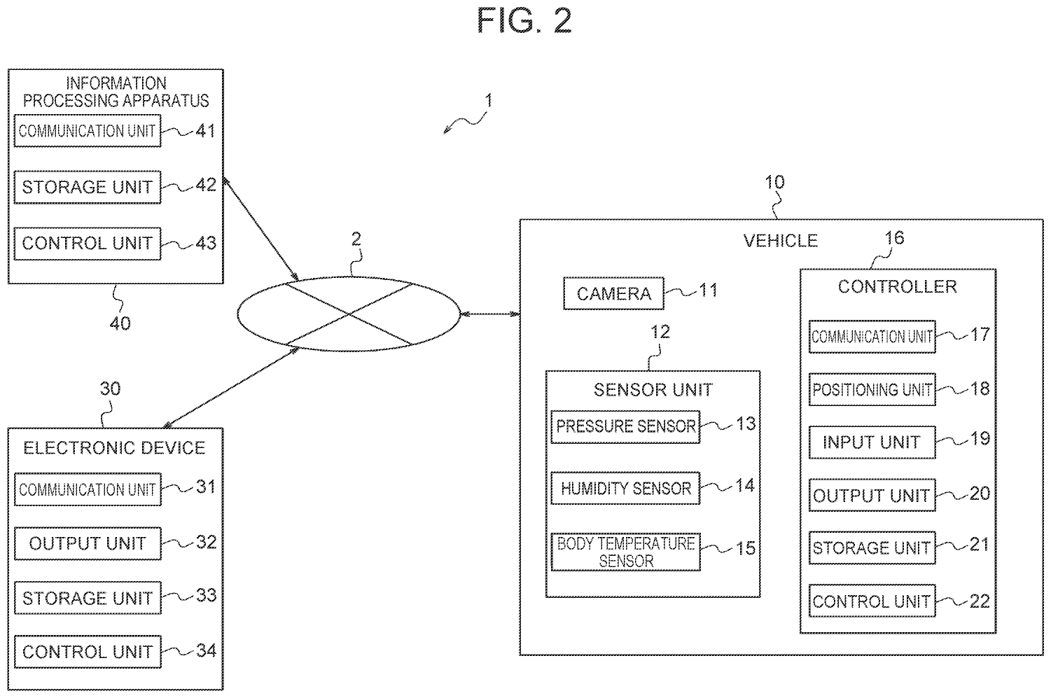

As shown in FIG. 2, the vehicle 10 includes a camera 11, a sensor unit 12, and a controller 16. The camera 11, the sensor unit 12, and the controller 16 are communicably connected with each other through, for example, an in-vehicle network such as a controller area network (CAN) or an exclusive line.

The camera 11 can be configured to include an imaging optical system and an imaging device. The camera 11 is located on a place where the eyes of the driver X can be imaged as a photographic subject. For example, as shown in FIG. 1, the camera 11 is located on the dashboard of the vehicle 10 as the place where the eyes of the driver X can be imaged.

The camera 11 generates an imaged image of the eyes of the driver X under the control of the controller 16. The camera 11 outputs data on the generated imaged image to the controller 16 under the control of the controller 16. The camera 11 may execute imaging at any frame rate under the control of the controller 16.

The sensor unit 12 includes a pressure sensor 13, a humidity sensor 14, and a body temperature sensor 15. However, the sensors included in the sensor unit 12 are not limited to these sensors. The sensor unit 12 may include any sensor capable of detecting information that indicates at least one state out of the state in a vehicle cabin of the vehicle 10 and the state of the driver X.

The pressure sensor 13 is located on the place where the pressure applied to the steering wheel is detectable. The pressure sensor 13 is located, for example, on the steering wheel as shown in FIG. 1. The pressure sensor 13 detects the pressure applied to the steering wheel under the control of the controller 16. The pressure sensor 13 can detect gripping force of the driver X gripping the steering wheel by detecting the pressure applied to the steering wheel. The pressure sensor 13 outputs information on the detected pressure to the controller 16 under the control of the controller 16.

The humidity sensor 14 is located on the place where perspiration of the driver X is detectable as humidity. The humidity sensor 14 is located, for example, on a driver's seat as shown in FIG. 1. The humidity sensor 14 is located on the driver's seat so that the perspiration of the driver X is detectable as humidity in the vicinity of the driver's seat. The humidity sensor 14 detects the humidity in the vicinity of the driver's seat under the control of the controller 16. The humidity sensor 14 outputs information on the detected humidity to the controller 16 under the control of the controller 16.

The body temperature sensor 15 is located on the place where the body temperature of the driver X is detectable. The body temperature sensor 15 may be a noncontact sensor, or may be a contact sensor. When, for example, the body temperature sensor 15 is a noncontact sensor, it is located on the driver's seat as shown in FIG. 1. The body temperature sensor 15 detects the body temperature of the driver X under the control of the controller 16. The body temperature sensor 15 outputs information on the detected body temperature of the driver X to the controller 16 under the control of the controller 16.

The controller 16 is a car navigation device, for example. The controller 16 includes a communication unit 17, a positioning unit 18, input unit 19, an output unit 20, a storage unit 21, and a control unit 22.

The communication unit 17 can be configured to include at least one communication module connectable with the network 2. The communication module is a communication module that conforms to communication standards, such as long term evolution (LTE), 4th generation (4G), or 5th generation (5G) communication standards.

The positioning unit 18 acquires information on the location of the vehicle 10 under the control of the control unit 22. The positioning unit 18 outputs the information on the location of the vehicle 10 to the control unit 22 under the control of the control unit 22. The positioning unit 18 can be configured to include a global positioning system (GPS) receiving module.

The input unit 19 can receive input from a user. The input unit 19 can be configured to include at least one input interface which can receive input from a user. The input interface may be a physical key, an electrostatic capacitance key, a pointing device, a touchscreen integrally provided with a display, a microphone, or the like.

The output unit 20 can output data. The output unit 20 can be configured to include at least one output interface which can output data. The output interface may be a display, a speaker, or the like. The display may be a liquid crystal display (LCD), an organic electro luminescence (EL) display, or the like.

The storage unit 21 can be configured to include at least one semiconductor memory, at least one magnetic memory, and at least one optical memory, or at least a combination of two types of these memories. For example, the semiconductor memory is a random access memory (RAM), or a read only memory (ROM). For example, the RAM is a static random access memory (SRAM) or a dynamic random access memory (DRAM). For example, the ROM is an electrically erasable programmable read only memory (EEPROM). The storage unit 21 may function as a main storage, an auxiliary storage, or a cache memory. The storage unit 21 stores data used for operation of the controller 16, and data obtained by operation of the controller 16. The storage unit 21 stores map information used for route guidance service when, for example, the controller 16 is a car navigation device.

The control unit 22 can be configured to include at least one processor and at least one specialized circuit, or a combination of these. The processor is a general-purpose processor, such as a central processing unit (CPU) or a graphics processing unit (GPU), or an exclusive processor specialized in specific processing. The specialized circuit is, for example, a field-programmable gate array (FPGA), or an application specific integrated circuit (ASIC). The control unit 22 can execute the processes related to operation of the controller 16, while controlling each unit of the controller 16. The control unit 22 has a clocking function to acquire current time.

The functions of the controller 16 may be implemented by executing a control program according to the present embodiment by a processor corresponding to the control unit 22. In other words, the functions of the controller 16 can be implemented by software. The control program can cause a computer to function as the controller 16 by causing the computer to execute operation of the controller 16. In other words, the computer can function as the controller 16 by executing the operation of the controller 16 in accordance with the control program.

In the present disclosure, the “program” can be recorded in a computer readable non-transitory recording medium. For example, the computer readable non-transitory recording medium is a magnetic recording medium, an optical disk, an optical magnetic recording medium, or a ROM. For example, the program may be distributed by selling, transferring or lending a portable recording medium, such as a digital versatile disc (DVD), or a compact disc read only memory (CD-ROM), having the program recorded therein. The program may be stored in a storage of the server. The program stored in the storage of the server may be distributed through transfer to other computers. The program may be provided as a program product.

In the present disclosure, the “computer” can temporarily store a program recorded in the portable recording medium or a program transferred from the server in the main storage, for example. The computer can further read a program stored in the main storage by a processor, and can execute processes in accordance with the read program by the processor. The computer may read a program directly from the portable recording medium, and may execute the processes in accordance with the program. Whenever a program is transferred to the computer from the server, the computer may execute the processes in accordance with the received program in sequence. The computer may execute the process by what is called an application service provider (ASP) service which implements the functions only through execution instructions and result acquisition, without through transfer of the program from the server to the computer. The program may include those equivalent to the program which is information used for processing by an electronic calculator. For example, the data which does not constitute direct instructions to the computer although having the property of defining the processes of the computer corresponds to “those equivalent to the program”.

Some or all of the functions of the controller 16 may be implemented by a specialized circuit corresponding to the control unit 22. In short, some or all of the functions of the controller 16 may be implemented by hardware.

When the controller 16 is a car navigation device, the control unit 22 can receive input of a destination from the driver X with the input unit 19. The control unit 22 can execute route guidance to the driver X upon reception of the input of the destination. For example, the control unit 22 acquires current location information on the vehicle 10 with the positioning unit 18. The control unit 22 searches for a route to the destination based on the current location information on the vehicle 10, the location information on the destination, and the map information in the storage unit 21. The control unit 22 makes the output unit 20 output the information indicating the searched route to the destination.

The control unit 22 can acquire driving information on the driver X of the vehicle 10. The control unit 22 can transmit the acquired driving information on the driver X to the information processing apparatus 40 through the network 2 with the communication unit 17. The control unit 22 may transmit the driving information on the driver X to the information processing apparatus 40 together with information on date and time when the driving information on the driver X has been acquired.

The driving information on the driver X may include any information collected in the vehicle 10. Examples of the driving information on the driver X includes information shown below.

The driving information on the driver X may include information on a movement history of the vehicle 10. The control unit 22 may acquire the information on the movement history of the vehicle 10 by acquiring the location information on the vehicle 10 with the positioning unit 18 while the vehicle 10 is moving.

The driving information on the driver X may include location information on a destination reached by the driver X driving the vehicle 10. When the controller 16 is a car navigation device, the control unit 22 may acquire the location information on the destination by receiving input of the destination with the input unit 19. The control unit 22 may also acquire the location information on the destination by analyzing the information on the movement history of the vehicle 10.

The driving information on the driver X may include information on driving time of the driver X. The control unit 22 may acquire the information on the driving time of the driver X by measuring the time when the driver X drives the vehicle 10 with use of the clocking function.

The driving information on the driver X may include biological information on the driver X during driving as shown below.

The biological information on the driver X during driving may include data on an imaged image of the eyes of the driver X during driving. The control unit 22 may acquire the data on the imaged image of the eyes of the driver X during driving with the camera 11.

The biological information on the driver X during driving may include information on gripping force of the driver X gripping the steering wheel during driving. The control unit 22 may acquire the information on the gripping force of the driver X gripping the steering wheel during driving with the pressure sensor 13.

The biological information on the driver X during driving may include information on the humidity in the vicinity of the driver's seat. As described above, the perspiration of the driver X may be detected as humidity in the vicinity of the driver's seat. The information on the humidity in the vicinity of the driver's seat can be regarded as the information on the perspiration amount of the driver X during driving, i.e., the biological information on the driver X during driving. The control unit 22 may acquire the information on the humidity in the vicinity of the driver's seat with the humidity sensor 14 during driving of the driver X.

The biological information on the driver X during driving may include information on the body temperature of the driver X during driving. The control unit 22 may acquire the information on the body temperature of the driver X during driving with the body temperature sensor 15.

As shown in FIG. 2, the home electric appliance 30 includes a communication unit 31, an output unit 32, a storage unit 33, and a control unit 34.

The communication unit 31 can be configured to include at least one communication module connectable with the network 2. For example, the communication module is a communication module that conforms to communication standards, such as local area network (LAN) or wireless LAN communication standards. The communication unit 31 can be connected to the network 2 through a wired LAN or wireless LAN with the communication module.

The output unit 32 can output data. As in the case of the output unit 20, the output unit 32 can be configured to include at least one output interface which can output data.

As in the case of the configuration of the storage unit 21, the storage unit 33 can be configured to include at least one semiconductor memory, at least one magnetic memory, at least one optical memory, or a combination of at least two types of these memories. The storage unit 33 may function as a main storage, an auxiliary storage, or a cache memory. The storage unit 33 stores data used for operation of the home electric appliance 30, and data obtained by operation of the home electric appliance 30.

As in the case of the configuration of the control unit 22, the control unit 34 can be configured to include at least one processor and at least one specialized circuit, or a combination of these. The control unit 34 can execute the processes related to operation of the home electric appliance 30, while controlling each unit of the home electric appliance 30.

The functions of the home electric appliance 30 can be implemented by executing an appliance program according to the present embodiment by a processor corresponding to the control unit 34. In other words, the functions of the home electric appliance 30 can be implemented by software. The appliance program enables a computer to function as the home electric appliance 30 by causing the computer to execute operation of the home electric appliance 30. In other words, the computer can function as the home electric appliance 30 by executing the operation of the home electric appliance 30 in accordance with the appliance program.

Some or all of the functions of the home electric appliance 30 may be implemented by a specialized circuit corresponding to the control unit 34. In short, some or all of the functions of the home electric appliance 30 may be implemented by hardware.

The control unit 34 can receive information on the recipe of a dish from the information processing apparatus 40 through the network 2 with the communication unit 31. The control unit 34 can make the output unit 32 output the information on the received recipe of the dish. For example, when the output unit 32 is a display, the control unit 34 makes the output unit 32 display the information on the received recipe of the dish. When the output unit 32 outputs the information on the recipe of a dish, the driver X can refer to the recipe of the dish after driving the vehicle 10 to go home.

As shown in FIG. 2, the information processing apparatus 40 includes a communication unit 41, a storage unit 42, and a control unit 43.

As in the case of the communication unit 31, the communication unit 41 can be configured to include at least one communication module connectable with the network 2. For example, the communication unit 41 can be connected to the network 2 through a wired LAN or a wireless LAN with a communication module that conforms to standards, such as wired LAN and wireless LAN standards.

As in the case of the configuration of the storage unit 21, the storage unit 42 can be configured to include at least one semiconductor memory, at least one magnetic memory, at least one optical memory, or a combination of at least two types of these memories. The storage unit 42 may function as a main storage, an auxiliary storage, or a cache memory. The storage unit 42 stores data used for operation of the information processing apparatus 40, and data obtained by operation of the information processing apparatus 40.

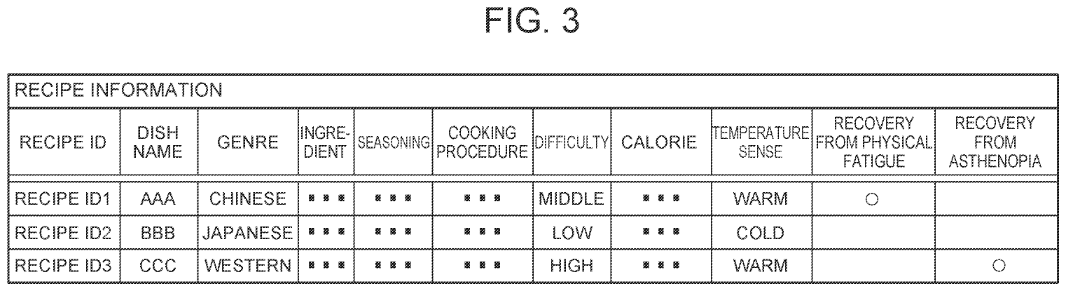

The storage unit 42 stores, for example, information on recipes of the dishes as shown in FIG. 3. The storage unit 42 stores, for example, map information. The map information in the storage unit 42 may include location information on facilities, and information on the types of the facilities.

As in the case of the configuration of the control unit 22, the control unit 43 can be configured to include at least one processor, at least one specialized circuit, or a combination of these. The control unit 43 can execute the processes related to the operation of the information processing apparatus 40, while controlling each unit of the information processing apparatus 40.

The functions of the information processing apparatus 40 can be implemented by executing an information processing program according to the present embodiment by a processor corresponding to the control unit 43. In other words, the functions of the information processing apparatus 40 may be implemented by software. The information processing program enables a computer to function as the information processing apparatus 40 by causing the computer to executing the operation of the information processing apparatus 40. In other words, the computer can function as the information processing apparatus 40 by executing the operation of the information processing apparatus 40 in accordance with the information processing program.

Some or all of the functions of the information processing apparatus 40 may be implemented by a specialized circuit corresponding to the control unit 43. In short, some or all of the functions of the information processing apparatus 40 may be implemented by hardware.

### Recipe Determination Process

The control unit 43 can receive driving information on the driver X from the vehicle 10 through the network 2 with the communication unit 41. The control unit 43 can determine the recipe of a dish to be suggested to the driver X based on the received driving information on the driver X. The control unit 43 may determine the recipe to be suggested to the driver X based on the driving information on the driver X and the information on the recipes of the dishes as shown in FIG. 3.

As shown in FIG. 3, in the information on the recipes of the dishes, the recipes may each be distinguished by their identifiers. Recipes ID1, ID2, ID3 are identifiers of the recipes of different dishes. The identifiers of the recipes are each associated with information on a dish name, information on the genre of the dish, information on ingredients to be used, information on seasoning to be used, and information on cooking procedures. The information on cooking procedures can include information on cooking methods. For example, the recipes ID1, ID2, ID3 are associated with information on the dish names of AAA, BBB, CCC, respectively. The recipes ID1, ID2, ID3 are associated with the information on the genres of the dishes: Chinese; Japanese; and Western, respectively.

As shown in FIG. 3, the identifier of each recipe may be associated with difficulty. The difficulty may be an index indicating the level of difficulty in completing the cooking in accordance with the recipe. The difficulty is set to three levels including “high”, “middle”, and “low”, for example. The “high” difficulty indicates that the difficulty is higher than a preset reference level. The “middle” difficulty indicates that the difficulty is the same as the preset reference level. The “low” difficulty indicates that the difficulty is lower than the preset reference level. The reference level may be set as appropriate based on cooking experience, cooking technique, and the like, of general persons. The difficulty of recipes may be set as appropriate based on the number of the cooking procedures, the type of cooking method, and the like, included in the recipes. For example, the recipe ID1 is associated with the “middle” difficulty. The recipe ID2 is associated with the “low” difficulty. The recipe ID3 is associated with the “high” difficulty.

As shown in FIG. 3, the identifier of each recipe may be associated with information on the calorie of the corresponding dish. The identifier of each recipe may also be associated with a flag indicating a temperature sense that general persons feel for the dish. A “warm” flag is a flag indicating that general persons sense that the dish is warm. A “cold” flag is a flag indicating that general persons sense that the dish is cold. For example, the recipes ID1, ID3 are attached with the “warm” flag. The recipe ID2 is attached with the “cold” flag.

As shown in FIG. 3, in the information on the recipes of the dishes, the recipe of the dish that promotes recovery from physical fatigue may be attached with a flag indicating that the dish promotes recovery from physical fatigue. For example, the recipe ID1 is attached with the flag indicating that the dish promotes recovery from physical fatigue. The dish that promotes recovery from physical fatigue may be preset based on nutrients of the ingredients in the recipe of the dish.

As shown in FIG. 3, in the information on the recipes of the dishes, the recipe of the dish that promotes recovery from eye fatigue may be attached with a flag indicating that the dish promotes recovery from eye fatigue. For example, the recipe ID3 is attached with the flag indicating that the dish promotes recovery from eye fatigue. The dish that promotes recovery from eye fatigue may be preset based on nutrients of the ingredients in the recipe of the dish.

The control unit 43 may transmit the information on the determined recipe of the dish to the preset home electric appliance 30 through the network 2 with the communication unit 41. The control unit 43 may transmit to the home electric appliance 30 at least information on a dish name, information on the genre of a dish, information on ingredients to be used, information on seasoning to be used, and information on cooking procedures, included in the information on the recipe of the dish. The driver X may set in advance the home electric appliance 30 to which the control unit 43 transmits the information on the recipe of the dish. The driver X may use a terminal device, such as a smartphone, to set in advance the home electric appliance 30 to which the control unit 43 transmits the information on the recipe of the dish. For example, the driver X uses the terminal device to transmit information on the home electric appliance 30, to which the information on the recipe of the dish is to be output, to the information processing apparatus 40 through the network 2. The control unit 43 handles the information on the home electric appliance 30, which is transmitted from the terminal device and received in the communication unit 41 through the network 2 and to which the information on the recipe of the dish is to be output, as the information on the home electric appliance 30 to which the information on the recipe of the dish is transmitted.

Here, the control unit 43 may determine the recipe of the dish to be suggested to the driver X based on the driving information received from the vehicle 10 from the time of determining the recipe of the dish to the time a set period before the time of determining the recipe of the dish. In other words, the driving information on the driver X may be the information received from the time when the control unit 43 determines the recipe of the dish to the time a set period before the control unit 43 determines the recipe of the dish, the set period being set in advance. The set time may be set as appropriate in accordance with the type of the driving information to be used for determination of the recipe of the dish as will be described later. The driver X may set in advance the time of determining the recipe of the dish. The driver X may set in advance the time of determining the recipe of the dish, by using a terminal device, such as a smartphone. For example, the driver X uses the terminal device to transmit information on the time of determining the recipe of the dish to the information processing apparatus 40 through the network 2. The driver X transmits in advance the information on the time of determining the recipe of the dish to the information processing apparatus 40, and thereby sets in advance the time of determining the recipe of the dish. An example of a process for determining the recipe of the dish will be described below.

### Example 1

The driving information on the driver X may include location information on a destination. In this case, when the location of the destination approximately coincides with the location of a sport facility, the control unit 43 may determine as the recipe of the dish to be suggested to the driver X the recipe of a preset dish that promotes recovery from physical fatigue. The sport facility may be a facility where general persons can play some sports. The sport facility is, for example, a ski site, a bowling stadium, and the like. In an example 1, the set time may be set as appropriate based on the time after the vehicle 10 leaves home until the vehicle 10 goes back home. In the example 1, the set time is, for example, one day to two days.

The control unit 43 may determine whether the location of the destination approximately coincides with the location of the sport facility by referring to the map information in the storage unit 42. When determining that the location of the destination approximately coincides with the location of the sport facility, the control unit 43 may determine to suggest to the driver X the recipe of a dish attached with a flag indicating that the dish promotes recovery from physical fatigue in the recipe information as shown in FIG. 3. For example, the control unit 43 determines to suggest the recipe ID1 to the driver X. The control unit 43 also transmits information on the recipe associated with the recipe ID1 to the home electric appliance 30.

When the location of the destination approximately coincides with the location of the sport facility, there is a high possibility that the driver X has played some sports in the sport facility. When the driver X has played some sports, there is a high possibility that the driver X is physically fatigued. When the location of the destination approximately coincides with the location of the sport facility, the recipe of a dish that promotes recovery from physical fatigue is determined as the recipe of the dish to be suggested to the driver X. Accordingly, the information on the recipe of the dish that promotes recovery from physical fatigue can be output from the output unit 32 of the home electric appliance 30. After driving the vehicle 10 to go back home from the sport facility, the driver X can refer to the recipe of the dish that promotes recovery from physical fatigue through the output unit 32. The driver X can eat the dish cooked and completed in accordance with the recipe of the dish. With such configuration, it is possible to promote recovery of the driver X from physical fatigue. Therefore, according to the example 1, it is possible to suggest to the driver X the recipe of a dish suitable for the physical condition of the driver X.

### Example 2

The driving information on the driver X may include location information on a destination. In this case, when the location of the destination approximately coincides with the location of a sport facility, the control unit 43 may determine as the recipe of the dish to be suggested to the driver X the recipe of a dish that is lower in difficulty than a preset reference level. In the example 2, the set time may be the same as in the example 1.

The control unit 43 may determine, as in the example 1, whether the location of the destination approximately coincides with the location of the sport facility by referring to the map information in the storage unit 42. When determining that the location of the destination approximately coincides with the location of the sport facility, the control unit 43 may determine to suggest to the driver X the recipe of a dish having a “low” difficulty in the recipe information as shown in FIG. 3. For example, the control unit 43 determines to suggest the recipe ID2 to the driver X. The control unit 43 also transmits information on the recipe associated with the recipe ID2 to the home electric appliance 30.

When the location of the destination coincides approximately with the location of the sport facility, there is a high possibility that the driver X is physically fatigued. The driver X may feel reluctant to cook, when the driver X is physically fatigued. In the present embodiment, when the location of the destination approximately coincides with the location of the sport facility, the recipe of a dish with low difficulty may be determined as the recipe of the dish to be suggested to the driver X. As a result, the information on the recipe of the dish with low difficulty may be output from the output unit 32 of the home electric appliance 30. After driving the vehicle 10 to go back home from the sport facility, the driver X can refer to the recipe of the dish with low difficulty through the output unit 32. With such configuration, it is possible to reduce the possibility that the driver X feels reluctant to cook. Therefore, according to the example 2, it is possible to suggest to the driver X the recipe of a dish suitable for the physical condition of the driver X.

### Example 3

The driving information on the driver X may include location information on a destination. In this case, when the location of the destination approximately coincides with the location of a food theme park, the control unit 43 may determine as the recipe of the dish to be suggested to the driver X the recipe of a dish with a lower calorie than a preset reference calorie. The reference calorie may be an average of the calories that the driver X takes per meal. The food theme park may be a facility including a collection of specialty stores specialized in a preset theme. For example, the food theme park is a facility including a collection of specialty stores specialized in ramen noodles, a facility including a collection of specialty stores specialized in sweets, or the like. In an example 3, the set time may be set as appropriate based on the time after the vehicle 10 leaves home until the vehicle 10 goes back home. In the example 3, the set time is, for example, one day to two days.

The control unit 43 may determine, as in the example 1, whether the location of the destination approximately coincides with the location of the food theme park by referring to the map information in the storage unit 42. When determining that the location of the destination approximately coincides with the location of the theme park facility, the control unit 43 may determine to suggest to the driver X the recipe of a dish with a lower calorie than the reference calorie in the recipe information as shown in FIG. 3.

When the location of the destination approximately coincides with the location of the food theme park, there is a high possibility that the driver X has eaten more foods than usual in the food theme park. In this case, when the driver X eats foods with the same calorie as usual, after coming back home from the theme park, the total caloric amount that the driver X takes that day may exceed the total caloric amount that the driver X usually takes per day. When the location of the destination approximately coincides with the location of the food theme park, the recipe of a dish with a low calorie may be determined as the recipe of the dish to be suggested to the driver X. As a result, the information on the recipe of the dish with a low calorie may be output from the output unit 32 of the home electric appliance 30. After driving the vehicle 10 to go back home from the food theme park, the driver X can refer to the recipe of the dish with a low calorie through the output unit 32. The driver X can eat a dish cooked and completed in accordance with the recipe of the dish. With such configuration, it is possible to reduce the possibility that the total caloric amount that the driver X takes that day may exceed the total caloric amount that the driver X usually takes per day. Therefore, according to the example 3, it is possible to suggest to the driver X the recipe of a dish suitable for the physical condition of the driver X.

### Example 4

The driving information on the driver X may include information on a movement history of the vehicle 10. In this case, the control unit 43 may estimate the genre of the dishes that the driver X prefers by analyzing the information on the movement history of the vehicle 10. The control unit 43 may determine to suggest the recipe of a dish belonging to the estimated genre to the driver X. In the example 4, the set time may be set as appropriate based on the frequency of the driver X visiting restaurants using the vehicle 10. In the example 4, the set time is, for example, several months.

The control unit 43 may identify the restaurants that the driver X has visited using the vehicle 10 based on the information on the movement history of the vehicle 10, and the map information in the storage unit 42, and estimate the genres of dishes provided by the restaurants. The control unit 43 may estimate, as the genre of the dish that the driver X prefers, the genre of the dish with a largest count among the estimated genres of the dishes. In the recipe information as shown in FIG. 3, the control unit 43 may determine as the recipe of the dish to be suggested to the driver X the recipe of a dish belonging to the genre of the dish that the driver X prefers. For example, when estimating that the genre of the dish that the driver X prefers is Japanese, the control unit 43 determines to suggest the recipe ID2 to the driver X. With such configuration, the driver X can refer to the recipe of a favorite dish. Therefore, according to the example 4, it is possible to suggest to the driver X the recipes of the dishes corresponding to the preference of the driver X.

### Example 5

The driving information on the driver X can include biological information on the driver X during driving. In this case, the control unit 43 may determine the recipe of the dish to be suggested to the driver X by analyzing the biological information on the driver X during driving. With such configuration, it is possible to suggest to the driver X the recipe of a dish suitable for the physical condition of the driver X. In the example 5, the set time may be set as appropriate based on the time when the biological information can change. Hereinafter, specific examples of the example 5 will be described in examples 6 to 10.

### Example 6

The biological information on the driver X during driving can include data on an imaged image of the eyes of the driver X during driving. In this case, the control unit 43 may detect eye fatigue of the driver X by analyzing the data on the imaged image. For example, the control unit 43 estimates a fatigue degree of the eyes of the driver X by analyzing the data on the imaged image. When the estimated fatigue degree exceeds a preset reference degree, the control unit 43 detects that the driver X has eye fatigue. As the degree of fatigue, any index indicating the degree of eye fatigue may be used. The reference degree may be set as appropriate based on the time when the physical condition of the driver X is normal. The reference degree is, for example, the degree of fatigue at the time when the physical condition of the driver X is normal. In the example 6, the set time may be set as appropriate based on the time when the state of the eyes of the driver X may change. In the example 6, the set time is, for example, one day to two days.

When the eye fatigue of the driver X is detected, the control unit 43 may determine as the recipe of the dish to be suggested to the driver X the recipe of a preset dish that promotes recovery from eye fatigue. When the eye fatigue of the driver X is detected, the control unit 43 may determine to suggest to the driver X the recipe of a dish attached with a flag indicating that the recipe of the dish promotes recovery from the eye fatigue in the recipe information as shown in FIG. 3. For example, the control unit 43 determines to suggest the recipe ID3 to the driver X. The control unit 43 also transmits information on the recipe associated with the recipe ID3 to the home electric appliance 30. With such configuration, since the driver X can eat the dish that promotes recovery from eye fatigue, it is possible to promote recovery of the driver X from eye fatigue. Therefore, according to the example 6, it is possible to suggest to the driver X the recipe of a dish suitable for the state of the eyes of the driver X.

### Example 7

The biological information on the driver X during driving may include information on gripping force of the driver X gripping the steering wheel of the vehicle 10. In this case, when the gripping force of the driver X gripping the steering wheel is weaker than a preset reference value, the control unit 43 may determine as the recipe of the dish to be suggested to the driver X of the vehicle 10 the recipe of a preset dish that promotes recovery from physical fatigue. The reference value may be an average of the gripping force of the driver X gripping the steering wheel. In the example 7, the set time may be set as appropriate based on the time when the gripping force of the driver X gripping the steering wheel can change. In the example 7, the set time is, for example, one day to two days.

When the gripping force of the driver X gripping the steering wheel is weaker than the preset reference value, the control unit 43 may determine, as in the example 1, to suggest to the driver X the recipe of a dish attached with a flag indicating that the dish promotes recovery from the physical fatigue in the recipe information as shown in FIG. 3.

When the gripping force of the driver X gripping the steering wheel is weaker than the preset reference value, there is a high possibility that the driver X is physically fatigued. When the gripping force of the driver X gripping the steering wheel is weaker than the preset reference value, the recipe of the dish to be suggested to the driver X is determined to be the recipe of the dish that promotes recovery from physical fatigue. As a result, as in the example 1, the driver X can eat the dish that promotes recovery from the physical fatigue. With such configuration, it is possible to promote recovery of the driver X from physical fatigue. Therefore, according to the example 7, it is possible to suggest to the driver X the recipe of a dish suitable for the physical condition of the driver X.

### Example 8

The biological information on the driver X during driving can include information on the humidity in the vicinity of the driver's seat of the vehicle 10. In this case, the control unit 43 may estimate a perspiration amount of the driver X by analyzing the information on the humidity in the vicinity of the driver's seat of the vehicle 10. In the example 8, the set time may be set as appropriate based on the time when the perspiration amount of the driver X can change. In the example 8, the set time is, for example, one day to two days.

When the estimated perspiration amount of the driver X is larger than a preset reference perspiration amount, the control unit 43 may determine as the recipe of the dish to be suggested to the driver X the recipe of a preset dish that promotes recovery from physical fatigue. The perspiration amount used as a reference may be a perspiration amount that is estimated such that the driver X may physically be fatigued when the driver X sweats for that perspiration amount.

When the estimated perspiration amount of the driver X is larger than the preset reference perspiration amount, the control unit 43 may determine to suggest to the driver X the recipe of a dish attached with a flag indicating that the dish promotes recovery from the physical fatigue in the recipe information as shown in FIG. 3. With such configuration, since the driver X can eat the dish that promotes recovery from physical fatigue, it is possible to promote recovery of the driver X from the physical fatigue. Therefore, according to the example 8, it is possible to suggest to the driver X the recipe of a dish suitable for the physical condition of the driver X.

### Example 9

The biological information on the driver X during driving may include information on the body temperature of the driver X during driving. In this case, when the body temperature of the driver X is higher than a preset reference body temperature, the control unit 43 may determine as the recipe of the dish to be suggested to the driver X the recipe of a preset cold dish. The reference body temperature may be an average temperature of the driver X. When the body temperature of the driver X is higher than the preset reference body temperature, the control unit 43 may determine to suggest to the driver X the recipe of a dish attached with a “cold” flag in the recipe information as shown in FIG. 3. For example, the control unit 43 determines to suggest the recipe ID2 to the driver X. In the example 9, the set time may be set as appropriate based on the time when the body temperature of the driver X can change. In the example 9, the set time is, for example, one day to two days.

When the driver X is kept in the state where the body temperature of the driver X is higher than the reference body temperature, the driver X may suffer heatstroke or the like and become sick. When the body temperature of the driver X is higher than the preset reference body temperature, the recipe of the dish to be suggested to the driver X is determined to be the recipe of a cold dish, so that the driver X can eat the cold dish. With such configuration, it is possible to reduce the possibility that the driver X becomes sick. Therefore, according to the example 9, it is possible to suggest to the driver X the recipe of a dish suitable for the physical condition of the driver X.

### Example 10

The biological information on the driver X during driving may include information on the body temperature of the driver X during driving. In this case, when the body temperature of the driver X is lower than the reference body temperature, the control unit 43 may determine as the recipe of the dish to be suggested to the driver X the recipe of a preset warm dish. When the body temperature of the driver X is lower than the preset reference body temperature, the control unit 43 may determine to suggest to the driver X the recipe of a dish attached with a “warm” flag in the recipe information as shown in FIG. 3. For example, the control unit 43 determines to suggest the recipe ID3 to the driver X. In the example 10, the set time may be the same as in the example 9.

When the driver X is kept in the state where the body temperature of the driver X is lower than the reference body temperature, the driver X may get cold or the like, and become sick. When the body temperature of the driver X is lower than the preset reference body temperature, the recipe of the dish to be suggested to the driver X is determined to be the recipe of a warm dish, so that the driver X can eat the warm dish. With such configuration, it is possible to reduce the possibility that the driver X becomes sick. Therefore, according to the example 10, it is possible to suggest to the driver X the recipe of a dish suitable for the physical condition of the driver X.

### Modification 1

The control unit 43 may determine the recipe of a dish to be suggested to the driver X and also determine to suggest increase or decrease of seasoning included in the recipe of the dish. The control unit 43 may determine to suggest increase or decrease of seasoning based on the driving information on the driver X.

When determining to suggest increase of seasoning, that the control unit 43 may transmit a notification of suggesting increase of seasoning together with the information on the recipe of a dish to the home electric appliance 30 through the network 2 with the communication unit 41. In the home electric appliance 30, the control unit 34 can receive the notification from the information processing apparatus 40 through the network 2 with the communication unit 31. In the home electric appliance 30, upon reception of the notification, the control unit 34 may output the information indicating suggestion of increase of seasoning from the output unit 32.

When determining to suggest decrease of seasoning, the control unit 43 may transmit a notification of suggesting decrease of seasoning together with the information on the recipe of a dish to the home electric appliance 30 through the network 2 with the communication unit 41. In the home electric appliance 30, the control unit 34 can receive the notification from the information processing apparatus 40 through the network 2 with the communication unit 31. In the home electric appliance 30, upon reception of the notification, the control unit 34 may output the information indicating suggestion of decrease of seasoning from the output unit 32.

In an example, the driving information on the driver X can include information indicating the humidity in the vicinity of the seat of the vehicle 10 as the biological information on the driver X during driving. In this case, the control unit 43 may estimate the perspiration amount of the driver X by analyzing the information on the humidity in the vicinity of the seat of the vehicle 10. When the estimated perspiration amount of the driver X is larger than a preset threshold, the control unit 43 may determine to suggest increase of an amount of salt, as the seasoning included in the determined recipe of the dish, from a reference salt amount in the recipe. The threshold may be set as appropriate based on the perspiration amount which may cause dehydration for general persons. When determining to suggest increase of the amount of salt as the seasoning, the control unit 43 may transmit to the home electric appliance 30 a notification indicating suggestion for increasing the amount of salt from the reference salt amount in the recipe. In the home electric appliance 30, upon reception of the notification, the control unit 34 may output from the output unit 32 the information indicating suggestion for increasing the amount of salt from the reference salt amount in the recipe. With such configuration, since the driver X can increase the amount of salt in cooking, it is possible to reduce the possibility of the driver X suffering dehydration.

### Modification 2

The control unit 43 may determine the recipe of a dish to be suggested to the driver X based on a combination of any type of information included in the driving information on the driver X. In an example, the control unit 43 may determine the recipe of the dish to be suggested to the driver X based on a combination of the location information on the destination and the information on the movement history of the vehicle 10. In this case, the control unit 43 may estimate the genre of a dish that the driver X prefers by analyzing the information on the movement history of the vehicle 10. When the location of the destination approximately coincides with the location of a sport facility, the control unit 43 may further select the recipe of a dish that promotes recovery from physical fatigue from the recipes of the dishes belonging to the estimated genre of the dish that the driver X prefers. The control unit 43 may determine, as the recipe of the dish to be suggested to the driver X, the recipe of the dish that promotes recovery from physical fatigue, selected from the genre of the dish that the driver X prefers.

### Operation of Suggestion System

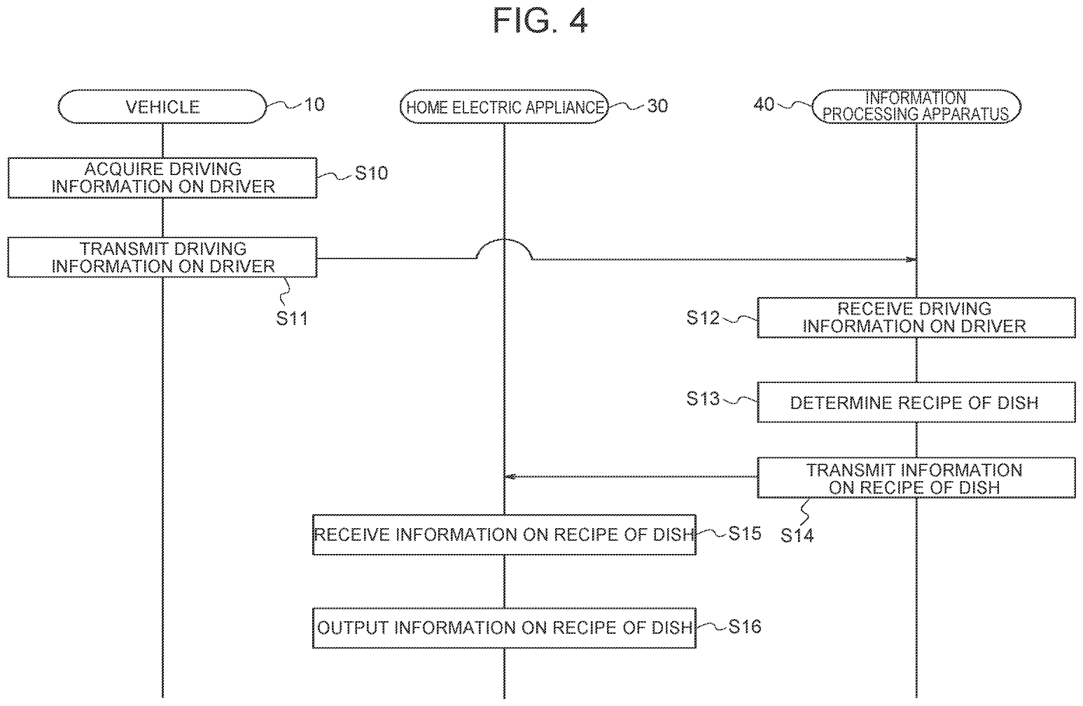

An example of the operation of the suggestion system 1 shown in FIG. 1 will be described with reference to FIG. 4. The operation corresponds to an example of a suggestion method according to the present embodiment.

In the vehicle 10, the control unit 22 of the controller 16 acquires driving information on the driver X of the vehicle 10 (step S10). In the vehicle 10, the control unit 22 of the controller 16 transmits the acquired driving information to the information processing apparatus 40 through the network 2 with the communication unit 17 of the controller 16 (step 511).

In the information processing apparatus 40, the control unit 43 receives the driving information on the driver X from the vehicle 10 through the network 2 with the communication unit 41 (step S12). The control unit 43 determines the recipe of a dish to be suggested to the driver X based on the received driving information on the driver X (step S13). The control unit 43 transmits the information on the determined recipe of the dish to the preset home electric appliance 30 through the network 2 with the communication unit 41 (step S14).

In the home electric appliance 30, the control unit 34 receives the information on the recipe of the dish from the information processing apparatus 40 through the network 2 with the communication unit 31 (step S15). The control unit 34 makes the output unit 32 output the information on the received recipe of the dish (step S16).

Thus, in the suggestion system 1, the information processing apparatus 40 determines the recipe of the dish to be suggested to the driver X based on the driving information on the driver X. With such configuration, it is possible to suggest to the driver X the recipe of a dish suitable for the driver X.

The present disclosure is not limited to the embodiment disclosed. For example, a plurality of blocks described in the block diagrams may be combined, or one block may be divided into a plurality of blocks. Instead of chronologically executing a plurality of steps stated in the flowchart in accordance with the description, the steps may be executed in parallel or in different order in accordance with the performance of the devices that execute the respective steps or in accordance with the need. Further modifications are possible without departing from the scope of the present disclosure.

For example, in the embodiment disclosed, the controller 16 as shown in FIG. 2 is described as a car navigation device as an example. However, the controller 16 is not limited to the car navigation device. The controller 16 may be a device designed exclusively for execution of the aforementioned processes, or may be a general-purpose terminal device, such as a smartphone.

For example, in the embodiment disclosed, the information processing apparatus 40 as shown in FIG. 1 is described as a server. However, the information processing apparatus 40 is not limited to the server. The information processing apparatus 40 may be a home energy management system (HEMS), or the like, installed in the home of the driver X.

For example, in the embodiment disclosed, the information processing apparatus 40 is described to determine the recipe of a dish to be suggested to the driver X. However, the apparatus that determines the recipe of the dish to be suggested to the driver X is not limited to the information processing apparatus 40. The apparatus that determines the recipe of the dish to be suggested to the driver X may be any apparatus. The apparatus that determines the recipe of the dish to be suggested to the driver X may be, for example, the controller 16. In this case, in the controller 16, the control unit 22 may determine the recipe of the dish to be suggested to the driver X based on the driving information on the driver X. In the controller 16, the control unit 22 may transmit the information on the determined recipe of the dish to the home electric appliance 30 through the network 2 with the communication unit 17. The apparatus that determines the recipe of the dish to be suggested to the driver X is, for example, the home electric appliance 30. In this case, in the controller 16, the control unit 22 may transmit the driving information on the driver X to the home electric appliance 30 through the network 2 with the communication unit 17. In the home electric appliance 30, the control unit 34 may receive the driving information on the driver X from the vehicle 10 from the controller 16 through the network 2 with the communication unit 31. The control unit 34 may determine the recipe of a dish be suggested to the driver X based on the received driving information on the driver X.

What is claimed is:

1. An information processing apparatus, comprising:
a communication unit; and
a control unit configured to determine a recipe of a dish to be suggested to a driver of a vehicle based on driving information on the driver of the vehicle received with the communication unit.

2. The information processing apparatus according to claim 1, wherein:
the driving information includes location information on a destination reached by the driver driving the vehicle; and
the control unit is configured to determine as the recipe of the dish to be suggested to the driver of the vehicle the recipe of a preset dish that promotes recovery from physical fatigue, when a location of the destination approximately coincides with a location of a sport facility.

3. The information processing apparatus according to claim 1, wherein:
the driving information includes location information on a destination reached by the driver driving the vehicle; and
the control unit is configured to determine as the recipe of the dish to be suggested to the driver of the vehicle the recipe of a preset dish that is lower in difficulty level than a reference level, when a location of the destination approximately coincides with a location of a sport facility.

4. The information processing apparatus according to claim 1, wherein:
the driving information includes location information on a destination reached by the driver driving the vehicle; and
the control unit is configured to determine as the recipe of the dish to be suggested to the driver of the vehicle the recipe of a preset dish that is lower in calorie than a reference calorie, when a location of the destination approximately coincides with a location of a food theme park.

5. The information processing apparatus according to claim 1, wherein:
the driving information includes information on a movement history of the vehicle; and
the control unit is configured to estimate a genre of a dish preferred by the driver by analyzing the information on the movement history of the vehicle, and determine as the recipe of the dish to be suggested to the driver of the vehicle the recipe of a dish belonging to the genre.

6. The information processing apparatus according to claim 1, wherein:
the driving information includes biological information on the driver during driving; and
the control unit is configured to determine the recipe of the dish to be suggested to the driver of the vehicle by analyzing the biological information.

7. The information processing apparatus according to claim 6, wherein:
the biological information includes data on an imaged image of eyes of the driver during driving; and
when the control unit detects eye fatigue of the drivers by analyzing the imaged image, the control unit is configured to determine as the recipe of the dish to be suggested to the driver of the vehicle a preset recipe that promotes recovery from the eye fatigue.

8. The information processing apparatus according to claim 6, wherein:
the biological information includes information on gripping force of the driver gripping a steering wheel of the vehicle; and
the control unit is configured to determine as the recipe of the dish to be suggested to the driver of the vehicle a preset recipe that promotes recovery from physical fatigue, when the gripping force is weaker than a preset reference value.

9. The information processing apparatus according to claim 6, wherein:
the biological information includes information indicating a humidity in a vicinity of a seat of the vehicle; and
the control unit is configured to
estimate a perspiration amount of the driver by analyzing the information indicating the humidity in the vicinity of the seat of the vehicle, and
determine as the recipe of the dish to be suggested to the driver of the vehicle a preset recipe that promotes recovery from physical fatigue, when the estimated perspiration amount of the driver is larger than a preset reference value.

10. The information processing apparatus according to claim 7, wherein:
the biological information includes information on a body temperature of the driver; and
the control unit is configured to determine as the recipe of the dish to be suggested to the driver of the vehicle the recipe of a preset cold dish, when the body temperature is higher than a preset reference body temperature.

11. The information processing apparatus according to claim 6, wherein:
the biological information includes information on a body temperature of the driver; and
the control unit is configured to determine as the recipe of the dish to be suggested to the driver of the vehicle the recipe of a preset warm dish, when the body temperature is lower than a preset reference body temperature.

12. The information processing apparatus according to claim 1, wherein the control unit is configured to determine the recipe of the dish to be suggested to the driver of the vehicle, and also determine to suggest increase or decrease of seasoning included in the recipe.

13. The information processing apparatus according to claim 12, wherein:
the driving information includes information indicating a humidity in a vicinity of a seat of the vehicle; and
the control unit is configured to
estimate a perspiration amount of the driver by analyzing the information indicating the humidity in the vicinity of the seat of the vehicle, and
determine to suggest increase of an amount of salt, as the seasoning included in the recipe, from a reference salt amount in the recipe, when the estimated perspiration amount of the driver is larger than a preset threshold.

14. The information processing apparatus according to claim 1, wherein the driving information is information received from a time when the control unit determines the recipe of the dish to a time a set period before the control unit determines the recipe of the dish, the set period being set in advance.

15. The information processing apparatus according to claim 1, wherein the control unit transmits information on the determined recipe of the dish to a preset home electric appliance with the communication unit.

16. The information processing apparatus according to claim 15, wherein the home electric appliance is a refrigerator.

17. The information processing apparatus according to claim 15, wherein the home electric appliance is a microwave oven.

18. A suggestion system, comprising:
the information processing apparatus according to claim 15; and
the home electric appliance.

19. A program causing a computer to execute operation, the operation comprising:
receiving driving information on a driver of a vehicle; and
determining a recipe of a dish to be suggested to the driver of the vehicle based on the driving information on the driver of the vehicle.

20. A suggestion method, comprising:
by an information processing apparatus, receiving driving information on a driver of a vehicle; and
by the information processing apparatus, determining a recipe of a dish to be suggested to the driver of the vehicle based on the driving information on the driver of the vehicle.
